(19) United States
(12) Patent Application Publication
(10) Pub. No.: US 2023/0381424 A1
(43) Pub. Date: Nov. 30, 2023

(54) DRUG DELIVERY DEVICE ASSEMBLY AND ACCESSORY FOR DRUG DELIVERY DEVICE

(71) Applicant: AMGEN INC., Thousand Oaks, CA (US)

(72) Inventors: Carsten Bech, Thousand Oaks, CA (US); Jakob Halkjaer Pedersen, Frederiksberg (DK); Joshua Jay Dudman, Copenhagen (DK); Adrianus Gerardus Verschuren, Frederiksberg (DK); Chiushun Dan, Thousand Oaks, CA (US)

(21) Appl. No.: 18/032,396

(22) PCT Filed: Nov. 1, 2021
(86) PCT No.: PCT/US21/57515

§ 371 Date: Apr. 18, 2023

Related U.S. Application Data
(60) Provisional application No. 63/109,607, filed on Nov. 4, 2020.

(51) Int. Cl. A61M 5/32

(52) U.S. Cl. CPC A61M 5/3287; A61M 2205/581

(57) ABSTRACT

A drug delivery device assembly is provided, including an injector housing, a needle assembly, a drive assembly, and a dose feedback accessory. The injector housing may include a body with a proximal end, a distal end, a longitudinal axis extending between the proximal end and the distal end, and at least one window. The needle assembly is at least partially disposed within the body and may include a syringe barrel containing a medicament, a plunger stopper disposed in the syringe barrel, and a needle or a cannula. The drive assembly is at least partially disposed within the body and operably coupled with the needle assembly to urge the medicament through the needle or cannula during an injection sequence. The dose feedback accessory includes an accessory body configured to be selectively coupled with the injector housing and a feedback indicator configured to convey to a user information relating to an injection status event.

### CROSS-REFERENCE TO RELATED APPLICATIONS

Priority is claimed to U.S. Provisional Patent Application No. 63/109,607, filed Nov. 4, 2020, the entire contents of which are hereby incorporated by reference herein.

### FIELD OF THE INVENTION

This disclosure generally relates to a drug delivery device assembly and accessory for a drug delivery device. More particularly, the disclosure generally relates to an assembly and/or an accessory that includes an accessory body configured to be selectively coupled with the injector housing and a feedback indicator configured to convey to a user information relating to an injection status event.

### BACKGROUND

Drugs are administered to treat a variety of conditions and diseases. Autoinjectors (e.g., pen style autoinjectors) and on-body injectors offer several benefits in delivery of medicaments such as drugs and/or therapeutics. One of the benefits can include simplicity of use, as compared with traditional methods of delivery using, for example, conventional syringes. Autoinjectors may be used to deliver many different medicaments with varying viscosities and/or desired volumes.

It may be desirable for autoinjector users to inspect and/or observe certain characteristics of the autoinjector before and/or during use. Autoinjector instructions for use (“IFU”) may instruct, encourage, or recommend such inspection actions. For example, a user may desire to or be instructed to inspect a medicament via an autoinjector viewing window before using an autoinjector, such as to check for particulates, discoloration, or contaminants. A user may desire to or be instructed to observe the viewing window during the injection process, or at least before removing the autoinjector from contact with the patient's skin. More specifically, during the injection sequence the user may observe the decreasing volume of the medicament and the plunger stopper urging the medicament from the drug delivery device to determine when the injection is complete. These steps may reduce the likelihood of premature removal of the device from the delivery site, which can result in an incomplete dosage being delivered due to the drug spraying onto the skin surface.

It may be desirable for autoinjector users to maintain a particular force level and/or orientation during the injection. Autoinjector IFUs may instruct, encourage, or recommend such actions. For example, a user may desire to or be instructed to maintain a constant or baseline force and/or to maintain the autoinjector in an orientation perpendicular to the injection site during the injection sequence. These steps may increase the likelihood of a complete and successful injection and/or reduce pain or discomfort.

However, some autoinjector users may find it awkward, uncomfortable, or otherwise inconvenient to apply the desired force at the desired orientation while also observing the viewing window. For example, some users may prefer to aim their gaze in a direction generally parallel with and/or along a longitudinal axis of the autoinjector. Such an orientation may permit the user to generally ensure that the autoinjector remains perpendicular to the injection site. However, such an orientation may not be conducive to observing the viewing window, which is typically on the side of the autoinjector.

As described in more detail below, the present disclosure sets forth a drug delivery device assembly and an accessory for drug delivery devices, such as autoinjectors, that embodies advantageous alternatives to existing systems and methods, and that may address one or more of the challenges or needs mentioned herein, as well as provide other benefits and advantages.

### SUMMARY

A drug delivery device assembly is provided, including an injector housing, a needle assembly, a drive assembly, and a dose feedback accessory. The injector housing may include a body with a proximal end, a distal end, a longitudinal axis extending between the proximal end and the distal end, and at least one window. The needle assembly is at least partially disposed within the body and may include a syringe barrel containing a medicament, a plunger stopper disposed in the syringe barrel, and a needle or a cannula. The drive assembly is at least partially disposed within the body and operably coupled with the needle assembly to urge the medicament through the needle or cannula during an injection sequence. The dose feedback accessory includes an accessory body configured to be selectively coupled with the injector housing and a feedback indicator configured to convey to a user information relating to an injection status event.

The accessory body may be configured to snap on to the injector housing and/or to snap off of the injector housing. The accessory body may include a pair of arms configured to snap on to the injector housing.

The accessory body may be configured to slide on to the injector housing and/or to slide off of the injector housing. The accessory body may include an opening configured to receive and selectively couple with at least a portion of the injector housing.

The dose feedback accessory may further include a sensor configured to detect the injection status event, and the sensor may be configured to detect a movement or a position of the plunger stopper.

The assembly may include an actuator operably coupled with the drive assembly, and the sensor may be configured to detect a movement or a position of the actuator.

The feedback indicator may include at least one light source that signifies a movement or a position of the plunger stopper. The feedback indicator may include a plurality of light sources that each signify a movement or a position of the plunger stopper. The plurality of light sources may be generally aligned with the window. The plurality of light sources may provide an injection completion countdown.

The feedback indicator may include a speaker for generating an audible signal. The audible signal may include an injection completion countdown. The audible signal may include operation instructions.

An accessory for a drug delivery device is also provided, including an accessory body configured to be selectively coupled with an injector and a feedback indicator configured to convey to a user information relating to an injection status event.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description.

FIG. 1 is a front view of an exemplary drug delivery device that may be utilized with aspects of the present disclosure;

FIG. 2A is a front view of an exemplary drug delivery device assembly according to aspects of the present disclosure, including an injector having a window and an accessory having a feedback indicator to convey to a user information relating to the injection status;

FIG. 2B is a perspective view of the assembly shown in FIG. 2A, where the feedback indicator indicates that the injector is in the ready state;

FIG. 2C is a perspective view of the assembly shown in FIG. 2A, where the feedback indicator indicates that the injector is in the active injection state;

FIG. 3A is a front view of another exemplary drug delivery device assembly according to aspects of the present disclosure, including an injector having a window and an accessory having a feedback indicator to convey to a user information relating to the injection status;

FIG. 3B is a perspective view of the assembly shown in FIG. 3A, where the feedback indicator indicates that the injector is in the active injection state;

FIG. 3C is a perspective view of the assembly shown in FIG. 2A, where the feedback indicator indicates that the injector is in the completed injection state;

FIG. 4A is a side view of another exemplary drug delivery device assembly according to aspects of the present disclosure, including an injector having a window and an accessory having a feedback indicator to convey to a user information relating to the injection status;

FIG. 4B is a perspective view of the accessory shown in FIG. 4A;

FIG. 4C is another perspective view of the accessory shown in FIG. 4A;

FIG. 4D is a partial cross-sectional view of the assembly shown in FIG. 4A;

FIG. 4E is a partially exploded view of the assembly shown in FIG. 4A;

FIG. 5A is a side view of another exemplary drug delivery device assembly according to aspects of the present disclosure, including an injector having an actuator button and an accessory having a feedback indicator to convey to a user information relating to the injection status;

FIG. 5B is a perspective view of the accessory shown in FIG. 5A;

FIG. 5C is another perspective view of the accessory shown in FIG. 5A;

FIG. 5D is a partial cross-sectional view of the assembly shown in FIG. 5A; and

FIG. 5E is a partially exploded view of the assembly shown in FIG. 5A.

### DETAILED DESCRIPTION

Generally speaking, pursuant to these various embodiments, a drug delivery device (e.g., an autoinjector or other injector) is coupled with or used in conjunction with an accessory to provide user feedback. For example, the accessory may be coupled with the injector, configured to detect an injection status event, and to convey to a user information relating the injection status event.

The term “about” as used herein means +/−10% to the smallest significant digit. The term “patient's skin” may refer to the users uncovered, naked, or bare skin and/or the user's skin as it is covered by clothing, bandage, or other covering.

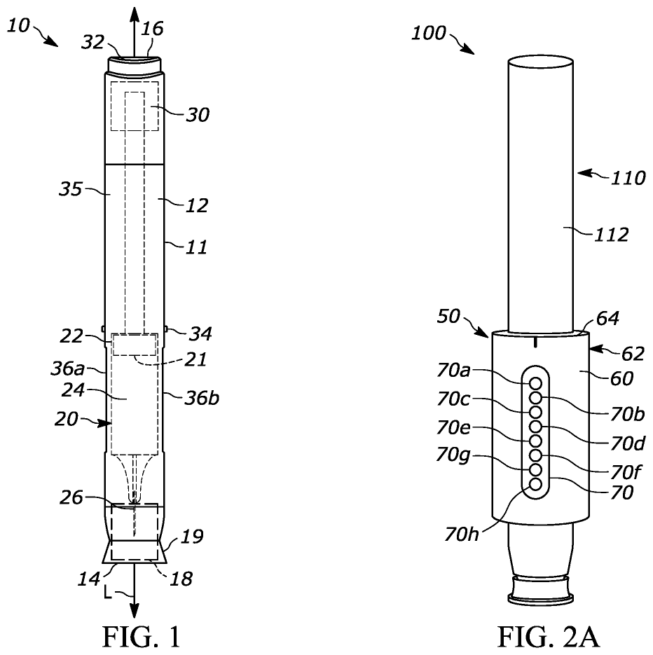

As illustrated in FIG. 1, an example injector 10 generally includes an injector housing 11 defining a housing 12 that includes a distal end 14, a proximal end 16, and a longitudinal axis L extending between the distal and proximal ends 14, 16. The injector 10 distal end 14 includes a generally cylindrical shaped needle shield 18 that assists with actuation of the injector 10 and a needle cap 19 that covers the needle shield 18 prior to use of the injector. A needle assembly 20 is at least partially disposed within the housing 12 at or near the distal end 14, and includes a syringe barrel 22 that contains a medicament 24, a plunger stopper 21 disposed within the syringe barrel 22, and a needle or a cannula 26 that is used to inject the medicament 24 into a patient. In the illustrated example, the needle or cannula 26 is initially positioned within the housing 12 prior to activation, and may protrude through an opening in the distal end 14 during drug delivery.

A drive assembly 30 is also at least partially disposed within the housing 12 and is operably coupled to the needle assembly 20. The drive assembly 30 may include an actuator button 32 positioned at or near the proximal end 16 of the housing 12 that initiates actuation of the drive assembly 30. In operation, a user removes the needle cap 19, places the needle shield 18 against the injection location (e.g., on their leg or their stomach), and actuates the actuator button 32. This actuation causes a drive mechanism (in the form of a spring, a motor, a hydraulic or pressurized mechanism, etc.) of the drive assembly 30 to exert a driving force on a portion of the needle assembly 20, such as the plunger stopper 21, that causes the needle or cannula 26 to be inserted through the opening of the housing 12 and into a patient and/or that further causes the medicament 24 to be urged from the syringe barrel 22, out the needle or cannula 26, and into the patient. In some versions, the patient may manually insert the needle or cannula 26, and actuation of the drive mechanism 30 only includes urging the plunger stopper 21 in the distal direction thereby causing the medicament 24 to be urged from the syringe barrel 22, out the needle or cannula 26, and to the patient. The injector 10 may not include an actuator button and may instead be activated by movement of the needle shield 18 alone, rather than an actuator button plus movement of the needle shield.

The injector 10 may include any number of additional features and components that may assist and/or enhance the functionality of the device. In the illustrated example, one or more knobs 34 project from an outer surface 35 of the housing 12 as an anti-roll feature. The one or more knobs 34 may be integrally formed with the housing 12 of the injector 10 or may be coupled to the outer surface 35 by welding, adhesive, or by another adhering method. Additionally, a viewing window 36 positioned at or near the syringe barrel 22 provides a visual indication of the remaining quantity of drug during administration. The needle cap 19 shields the needle 26 and prevents unintentional activation of the injector 10 and deployment of the needle or cannula 26. The needle shield 18 acts to unlock or initiate the injection when the needle shield 18 is pressed against a patient's skin. The activation of the drive assembly 30 requires a specific force to be applied to the needle shield 18 of the injector 10 and that force is transferred to the users skin. In other examples, the injector 10 may additionally include one or more electronic modules that are coupled to the housing 12, the needle assembly 20, the drive assembly 30, and/or any other components of the injector 10. Further, the injector 10 may also include any number of safety mechanisms such as a retraction mechanism, damping mechanism, and the like.

The present example of the drug delivery device 10 takes the form of an autoinjector or pen-type injector, and, as such, may be held in the hand of the user over the duration of drug delivery. The drug delivery device 10 may be suitable for self-administration by a patient or for administration by caregiver or a formally trained healthcare provider (e.g., a doctor or nurse). However, various implementations and configurations of the drug delivery device 10 are possible. In other examples, the drug delivery device 10 may be configured as a multiple-use reusable injector.

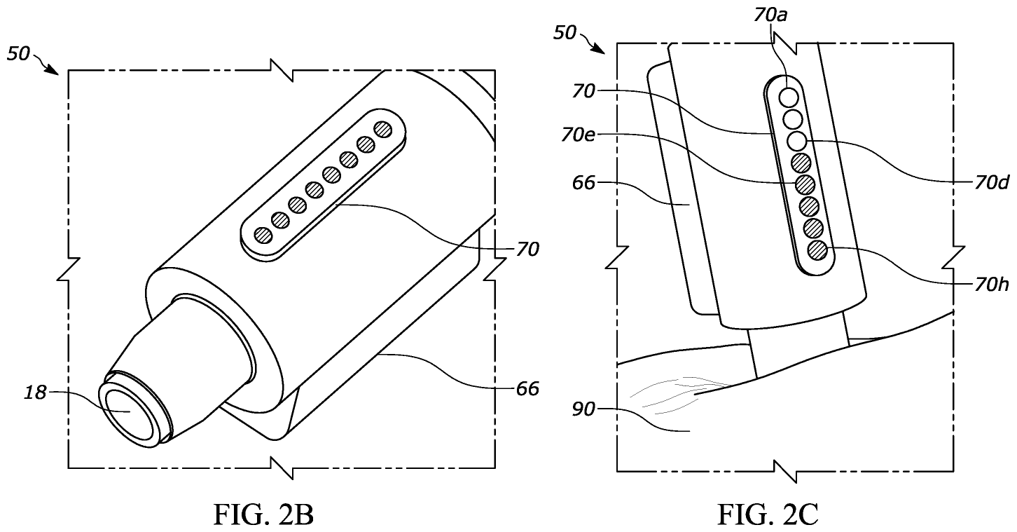

FIG. 2A shows a drug delivery device assembly 100, including an injector 110 and an accessory 50 according to aspects of the present disclosure. The accessory 50 includes an accessory body 60 configured to be selectively coupled with the injector housing 112 and a feedback indicator 70 configured to convey to a user information relating to an injection status event. The term injection status event may refer to one or more of a status, an event, a condition, a stage, including but not limited to an indication that the injector is ready for injection, that the injector is in the process of injecting, or that the injection is complete.

The accessory body 60 may be a generally cylindrical sleeve 62 with a generally circular opening 64 configured to receive the injector housing 112. As a more specific example, the opening 64 may have a similar or same shape as the received portion of the injector housing 112 (e.g., circular) and may be slightly larger or equal in diameter than the received portion of the injector housing 112 so that the body 60 is able to slide on and slide off of the injector 110. The body 60 and/or the injector 110 may also include a coupling portion that locks, couples, or otherwise holds together the accessory body 60 and the injector 110. For example, the coupling portion may be a flexible arm on the body 60 that presses against the injector 110 to form a friction fit engagement. As another example, the coupling portion may be a flexible arm on the body 60 that is received within a slot formed in the injector 110 to form a friction fit engagement. As yet another example, the coupling portion may be a protrusion, such as a rounded nub, that extends from the surface of the injector 110 and presses against the body 60 to form a friction fit engagement. As another example, the coupling portion may be a protrusion on the surface of the injector 110 that is received within a slot formed on the inner surface of the body 60, thereby forming a friction fit engagement. The body 60 may also be locked to the injector 110 via an additional step, such as by rotating the body 60 and injector 110 with respect to each other or engaging a movable locking arm.

The body 60 may be coupled with the injector 110 in a position and orientation such the sensor is aligned with a drug viewing window such as the window 36 shown in FIG. 1. As a more specific example, the body 60 includes a light source 66 (FIG. 2B) and that light source 66 is configured to be aligned with a first 36a (FIG. 1) of the two drug viewing windows such that the light shines through the first drug window 36a, through the medicament 24 in the syringe, through the second drug viewing window 36b, and then onto the feedback indicator 70. As a more specific example, the light source 66 may be light-emitting diode (LED) lights, organic light-emitting diode (OLED) lights, incandescent lights, neon lights, or any other suitable type of lights. The light source 66 may extend along much or all of the first window 36a, may be one or multiple lights, and may have any suitable shape and size. The light source 66 shown in FIG. 2B is shaped and sized to evenly shine light along the length of the second light window 36b. The feedback indicator 70 shown in the figures is aligned such that the feedback indicator 70 is able to receive all of the light that shines through the second window 36b and displays the light to the user. In other words, the feedback indicator displays the light to convey to a user information relating to an injection status event.

The term “injection status event” may refer to one or more of a status, an event, a condition, a stage, including but not limited to an indication that the injector is ready for injection, that the injector is in the process of injecting, that the injection is complete, or a position or a movement of a component of the injector. For example, the injection status may be related to the volume of the medicament 24 within the syringe barrel 22. As another example, the injection status may be a position of the plunger stopper 21, such as its position near the top (i.e., the proximal portion) of the window 36, as is shown in FIG. 1 or any other position within the syringe barrel 22. Additionally or alternatively, the injection status may be a movement of the plunger stopper 21, such as movement in the distal direction during the injection process. Additionally or alternatively, the injection status may be whether the injection process has started, is in process, and/or has been completed.

As mentioned above, the feedback indicator 70 is configured to convey to user information relating to an injection status event. The feedback indicator 70 may be a visual indicator, an auditory indicator, a tactile indicator, a combination of these types of indicators, or another suitable type of indicator. As a more specific example, the feedback indicator 70 shown in FIG. 2A includes a plurality of visual indicators, namely light diffusers. As an even more specific example, the feedback indicator 70 in FIG. 2A includes a series of light diffusers 70a, 70b, 70c, 70d, 70e, 70f, 70h, and 70h arranged in a generally linear fashion and generally aligned with the window 36. The light diffusers 70a-70h may be used to indicate movement or position of the plunger stopper 21. For example, the proximal most light diffuser 70a may be aligned with the top (i.e. the proximal portion) of the window 36 and the distal most light diffuser 70h may be aligned with the bottom of the window 36 and each of the respective light diffusers 70a-70h may turn on or off as the plunger stopper 21 is positioned adjacent to the same. The accessory 50 shown in FIGS. 2A-2C is spaced proximally far enough from the distal end of the injector 10 that the accessory does not obscure the needle shield 18 (FIG. 2B) and does not abut or interfere with the users injection spot 90 (FIG. 2C).

FIGS. 2B-2C show accessory, most prominently the light diffuser 70, during use. The light diffusers may be composed of optical fibers, or any other transparent or translucent material. The light diffusers 70a-70h shown in FIG. 2C are made optical fibers ground down to increase the light diffusion qualities there. As another example, if the material of the feedback indicator 70 is completely transparent and/or does not redirect or diffuse the light then it may be difficult for a user to view the feedback indicator from various angles. The light diffusers 70a-70h may be all on (e.g., lit up), as in FIG. 2B, at the beginning of an injection and/or before the injection starts. The light diffusers may “turn off” (e.g. become not lit) as the plunger stopper and/or plunger rod passes each of the respective light diffusers 70a-70h such that light diffusers 70a-70c are blocked by the distally-moving plunger stopper and/or plunger rod. In other words, during the injection process the light diffusers 70a-70h will give the appearance of lights being turned off as the injection sequence progresses. For example, the injection is partially finished in FIG. 2C.

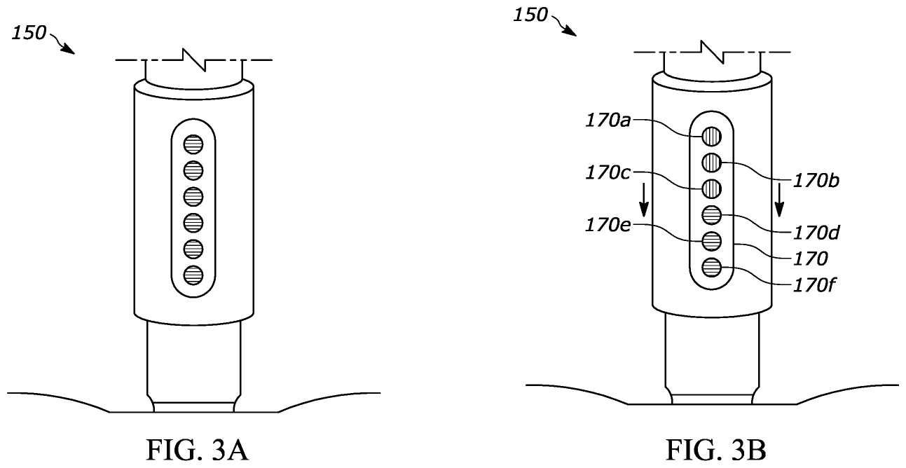

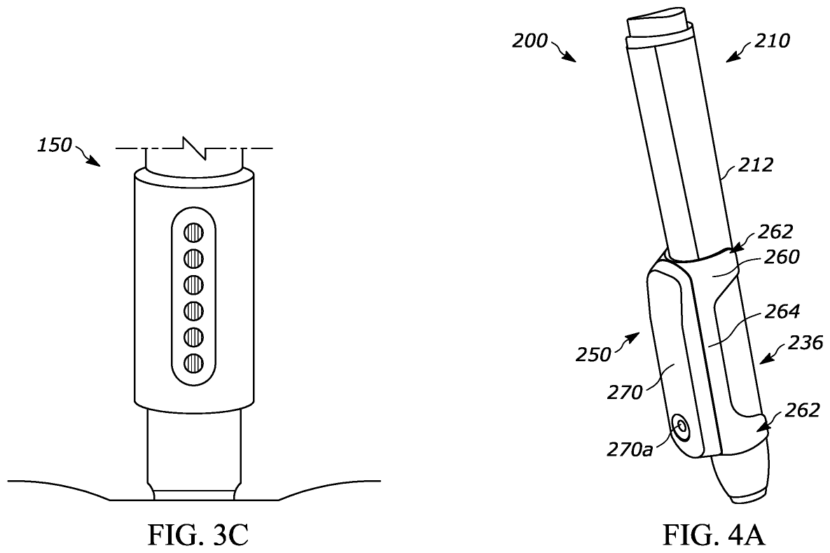

FIGS. 3A-3C show another embodiment of an accessory 150 including a feedback indicator 170 having six light diffusers 170a, 170b, 170c, 170d, 170e, 170f. The light diffusers 170a-170f may be all on (e.g., lit up), as in FIG. 3A, at the beginning of an injection and/or before the injection starts. The lights may turn off as the plunger stopper passes each of the respective light diffusers 170a-170f such that light diffusers 170a-170c are blocked (i.e., “turned off”) when the injection process is half finished, as in FIG. 3B, and all of the light diffusers 170a-170f are turned off when the injection process is complete, as in FIG. 3C. Alternative configurations may be utilized.

Although the accessory in FIGS. 2A-2C includes eight light diffusers 70a-70h and the accessory 150 in FIGS. 3A-3C includes six light diffusers 170a-170f, any suitable number of light diffusers may be utilized. For example, the accessory may include the number and individual sized light diffusers to correspond to the length of the window in the injector and/or the length of the syringe barrel. As another example, the accessory may include one single light diffuser extending along the length of the second window 36b. As yet another example, the accessory may include any suitable number of light diffusers, such as 1 light diffuser, 2 light diffusers, 4 light diffusers, 6 light diffusers, 8 light diffusers, 10 light diffusers, or another number of light diffusers. Additionally, while the above-described version includes a single light source, other variations may include a plurality of light sources to achieve a similar result.

The information conveyed to the user may be at least one or more of the following: the accessory is successfully coupled with the injector, the accessory is on, the injector is ready for the injection process, the injection process has begun, the injection process is in progress, the injection process is continuing, the injection process has stopped, the injection process is complete. The injector may signal this information via which light diffusers are lit, whether the light diffusers are changing from lit to unlit, or other characteristics.

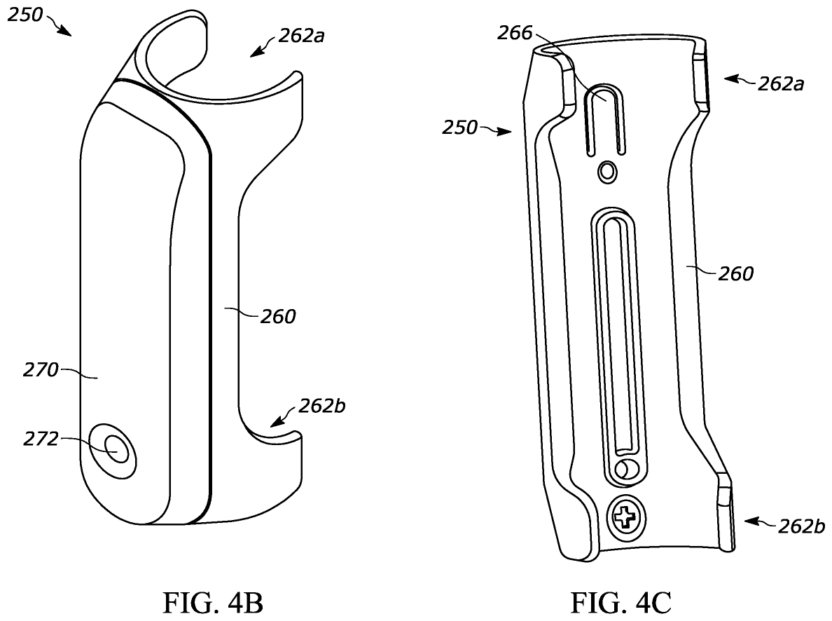

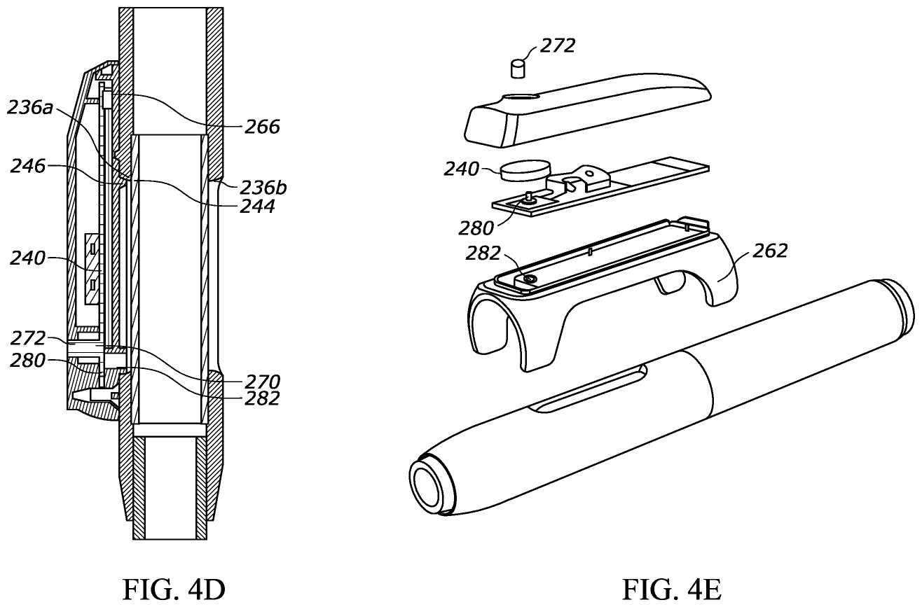

FIG. 4A shows a drug delivery device assembly 200, including an injector 210 and an accessory 250 according to aspects of the present disclosure. The accessory 250 includes an accessory body 260 configured to be selectively coupled with the injector housing 212, a sensor 280 configured to detect an injection status event, and a feedback indicator 270 configured to convey to a user information relating to the injection status event.

The accessory body 260 may include an elongated portion 264 and at least one pair of arms 262 configured to snap on to the injector housing 212. However, in some versions, due to the geometry of the arms 262, the same accessory body 260 may also be configured to slide on and slide off of the injector housing 212, as might be desired. As a more specific example, the accessory 250 shown in FIG. 4A includes two pairs of arms 262, a proximal pair and a distal pair. The arms 262 may be flexible such as to flex away from each other while the accessory 250 is being coupled with the injector 210, yet rigid enough to form a relatively snug connection between the accessory 250 and the injector 210. The elongated portion 264 may be aligned with and extend along one of the two drug windows 236. As a more specific example, the elongated portion 264 may be positioned with respect to a first drug window 236a (FIG. 4D) of the injector 200 such that the sensor 280 is aligned with the first drug window 236a. The second drug window 236b is visible on the other side of the injector 210 such that light is able to shine through drug window 236b to the accessory 250. More specifically, the two respective pairs of arms 262 are longitudinally spaced apart from each other such that the proximal pair of arms 262a (seen in FIG. 4C) are positioned just proximally of the proximal end of the drug window 236 and the distal arms 262b (seen in FIG. 4C) are positioned just distally of the distal end of the drug window 236, such as to not obstruct the drug window 236. The accessory 250 may include any suitable number of pairs of arms, such as 2, 4, 6, or any other suitable number. The accessory 250 may also include a ridge 246 (FIG. 4D) that abuts a portion of a window 236a to help align the accessory 250 and the injector 200.

The body 260 and/or the injector 210 may also include an additional coupling portion, in addition to the pairs of arms 262, that locks, couples, or otherwise holds together the accessory body 260 and the injector 210. For example, the coupling portion may be a protrusion on the surface of the injector 210 that is received within a slot formed on the inner surface of the body 260, thereby forming a friction fit engagement. The body 260 may also be locked to the injector 210 via an additional step, such as by rotating the body 260 and injector 210.

The body 260 may also include a contact switch 266 that is configured to selectively abut the housing 212 of the injector 210. As a more specific example, when the accessory 250 may be configured such that when the accessory 250 is properly coupled with the housing 212, the contact switch 266 is depressed. The contact switch 266 may be operatively coupled with the feedback indicator 270 such that the feedback indicator 270 is able to provide information to the user to indicate that the accessory 250 is properly coupled with the housing 212.

As shown in FIG. 4D, the sensor 280 may be a light sensor configured to measure light, detect light, and/or be sensitive to light. The sensor 280 may be connected to the first window 236a by a light channel 282 such that light entering the second light window 236b is able to shine through the syringe barrel, through the first window 236a, into the light channel 282, and be detected by the light sensor 280. As a further example, when the plunger stopper moves in the distal direction during the injection process then it may eventually become aligned with the light channel 282, thereby blocking light from entering the light channel 280. As a result, when the plunger stopper reaches a predetermined position, such as the distal end of the syringe, then the user is able to receive information regarding the end of injection event via the feedback indicator 270. The electrical components of the accessory 250 may be powered by a battery 240.

During use, the user may be notified of proper alignment and coupling between the accessory 250 and the injector 210 via the contact sensor 266 and the feedback indicator 270. For example, when a light 272 of the feedback indicator 270 may be a red shade. Once the injection begins and the plunger stopper passes a spot near the proximal portion of the window 236a, an upper portion of the light channel 282 may be temporarily obstructed by the plunger, thereby causing the feedback indicator 270 to cause the light to flash green. Once the injection is complete and the plunger passes the spot near the distal portion of the window 236a and the plunger stopper obstructs the distal light channel 282, then the light may change to solid green. In this example, the user is notified of all three states (ready to inject, start of the injection, and end of the injection). As another alternative configuration, the lights may have different color schemes, flashing statuses, or other features.

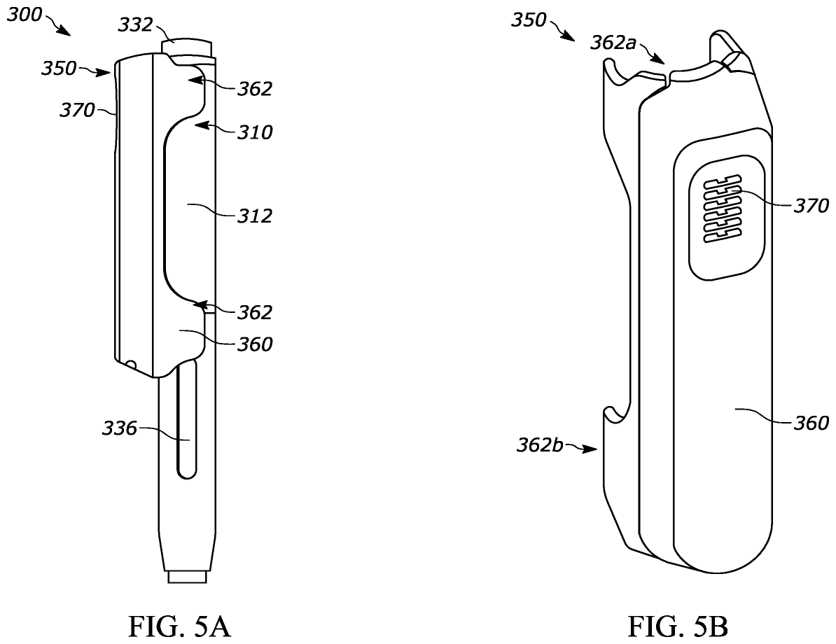

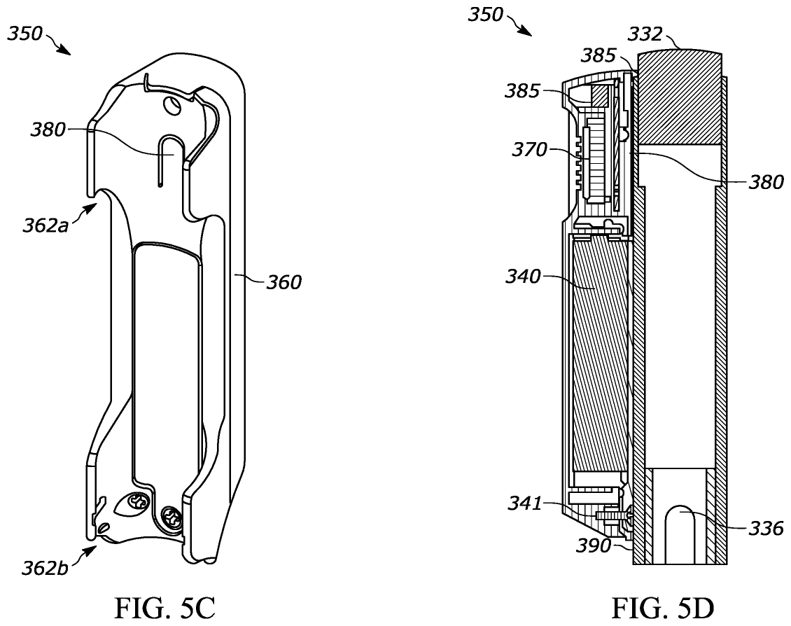

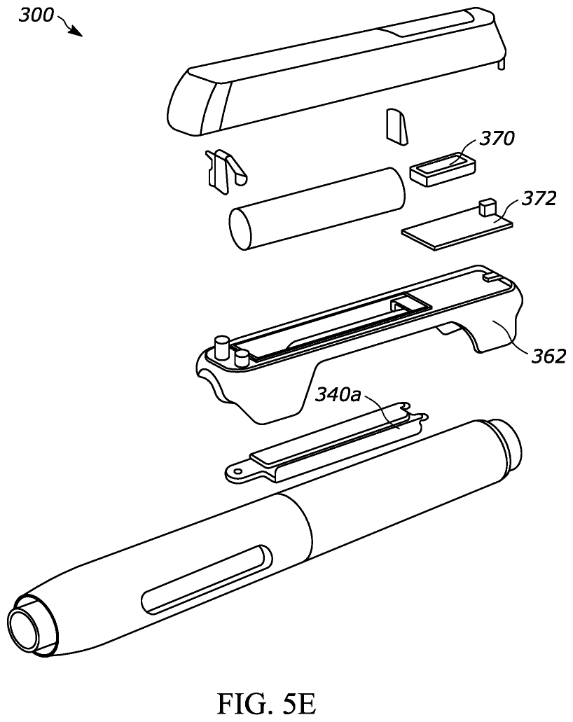

FIG. 5A shows a drug delivery device assembly 300, including an injector 310 and an accessory 350 according to aspects of the present disclosure. The accessory 350 includes an accessory body 360 configured to be selectively coupled with the injector housing 312, a sensor 380 (FIG. 5C) configured to detect an injection status event, and a feedback indicator 370 configured to convey to a user information relating to the injection status event.

The accessory body 360 may include an elongated portion and at least one pair of arms 362 configured to snap on to the injector housing 312. As a more specific example, the accessory 350 shown in FIG. 5A includes two pairs of arms 362, a proximal pair and a distal pair. The arms 362 may be flexible such as to flex away from each other while the accessory 350 is being coupled with the injector 310, yet rigid enough to form a relatively snug connection between the accessory 350 and the injector 310. The accessory 350 may be positioned such that a proximal portion of the accessory body 360 is positioned adjacent to an actuator button 332 and/or such that a distal portion of the accessory body 360 is positioned adjacent to the drug window 336. As a more specific example, the accessory 350 may include an actuator sensor 385 configured to detect an audible noise made by the actuator button 332 (FIG. 5D). For example, the actuator sensor 385 may be a microphone configured to detect the click noise made by the actuator button 332 when depressed. Additionally, some autoinjectors include components that generate an end of dose click and/or continuous or repeating click generated during the injection that may also be detected by the actuator sensor 385. As another example, the accessory may include a light sensor 390 configured to detect movement of the plunger stopper of the injector 300.

The body 360 may also include a contact sensor 380 that is configured to selectively abut the housing 312 of the injector 310. As a more specific example, when the accessory 350 may be configured such that when the accessory 350 is properly coupled with the housing 312, the contact switch 380 is depressed. The contact switch 380 may be operatively coupled with the feedback indicator 370 such that the feedback indicator 370 is able to provide information to the user to indicate that the accessory 350 is properly coupled with the housing 312. Additionally or alternatively, the feedback indicator 370 may be operatively coupled with the actuator sensor 385 and/or the light sensor 390.

The feedback indicator 370 may be configured to generate an audible signal. For example, the feedback indicator may be a speaker for generating the audible signal. In some versions, the feedback indicator 370 may include a speaker and also one or more light sources, as described above with earlier embodiments to provide multiple forms of feedback to the user. The audible signal may include operation instructions, such as “please remove the end cap and press the injector to the injector area.” The audible signal may also or alternatively include an injection countdown, such as an audible countdown from 10 to 1 for an injection that is expected to last 10 seconds.

During operation, when the user properly couples the accessory 350 to the injector 310, the contact switch 380 will be depressed and the feedback indicator 370 will indicate to the user that the accessory 350 and the injector 310 are coupled. For example, the feedback indicator 370 may make a predetermined audible sound or signal to the user. The audible sound or signal may be louder than the click made by depression of the actuator button 332 so as to assist users who have trouble hearing the same. The feedback indicator 370 may then instruct the user to take a step, such as apply the injector to the injection area and/or depress the actuator button 332. Finally, the feedback indicator 370 may utilize the actuator sensor 385 and/or the light sensor 390 to determine the stage of the injection process and provide feedback to the user regarding the same.

The accessories described herein may provide other types of feedback, such as haptic feedback that includes a vibration or other tactile feedback.

The syringe barrel may have a length of 45 to 85 mm, 60 to 65 mm, or another suitable length. The length of the syringe barrel is the length between the rear end to the outlet to which the needle is attached (but not including the needle, if present).

The syringe barrel may have an internal diameter of 4 to 6.5 mm. If the syringe has a nominal maximum fill volume of 1 ml, the internal diameter of the syringe barrel may be 5.5 to 6.5 mm. If the syringe has a nominal maximum fill volume of 0.5 ml, the internal diameter of the syringe barrel may be 4 to 5 mm.

The wall of the syringe barrel may have a thickness of at least 1 mm; about 1 to 3 mm; about 1.5 to 3 mm; or about 2.4 to 2.8 mm. Due to the thickness of the wall, the sterilizing gas is restricted or prevented from entering interior of the syringe, thereby minimizing or preventing contact with the liquid formulation contained within the prefilled syringe.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing di merization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™ Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG8 mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TI MP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vlIl (EGFRvIll) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device assembly comprising:
an injector housing having a body with a proximal end, a distal end, a longitudinal axis extending between the proximal end and the distal end, and at least one window;
a needle assembly at least partially disposed within the body, the needle assembly comprising a syringe barrel containing a medicament, a plunger stopper disposed in the syringe barrel, and a needle or a cannula;
a drive assembly at least partially disposed within the body and operably coupled with the needle assembly to urge the medicament through the needle or cannula during an injection sequence; and
a dose feedback accessory, including:
an accessory body configured to be selectively coupled with the injector housing; and
a feedback indicator configured to convey to a user information relating to an injection status event.

2. The drug delivery device assembly as in claim 1, wherein the dose feedback accessory further includes a sensor configured to detect the injection status event.

3. The drug delivery device assembly as in claim 1, wherein the accessory body is configured to snap on to the injector housing and/or snap off of the injector housing.

4. (canceled)

5. (canceled)

6. The drug delivery device assembly as in claim 1, wherein the accessory body is configured to slide on to the injector housing and/or slide off of the injector housing.

7. (canceled)

8. The drug delivery device assembly as in claim 1, wherein the accessory body includes an opening configured to receive and selectively couple with at least a portion of the injector housing.

9. The drug delivery device assembly as in claim 2, wherein the sensor is configured to detect a movement or a position of the plunger stopper.

10. The drug delivery device assembly as in claim 2, further comprising an actuator operably coupled with the drive assembly, and the sensor is configured to detect a movement or a position of the actuator.

11. The drug delivery device assembly as claim 1, wherein the feedback indicator includes (a) at least one light source that signifies a movement or a position of the plunger stopper, or (b) a plurality of light sources that each signify a movement or a position of the plunger stopper.

12. (canceled)

13. The drug delivery device assembly as in claim 11, wherein the feedback indicator includes (b) the plurality of light sources that each signify a movement or a position of the plunger stopper and the plurality of light sources are generally aligned with the window.

14. The drug delivery device assembly as in claim 11 or 13, wherein the feedback indicator includes (b) the plurality of light sources that each signify a movement or a position of the plunger stopper and the plurality of light sources provide an injection completion countdown.

15. The drug delivery device assembly as in claim 1, wherein the feedback indicator includes a speaker for generating an audible signal, wherein the audible signal includes (a) an injection completion countdown and/or (b) operation instructions.

16. (canceled)

17. (canceled)

18. An accessory for a drug delivery device, comprising:
an accessory body configured to be selectively coupled with an injector; and
a feedback indicator configured to convey to a user information relating to an injection status event.

19. The accessory as in claim 18, wherein the feedback indicator comprises a sensor configured to detect an injection status event of the drug delivery device.

20. The accessory as in claim 18, wherein the accessory body is configured to snap on to the drug delivery device and/or off of the drug delivery device.

21. (canceled)

22. (canceled)

23. The accessory as in claim 18, wherein the accessory body is configured to slide on to the drug delivery device and/or off of the drug delivery device.

24. (canceled)

25. The accessory as in claim 18, wherein the accessory body includes an opening configured to receive and selectively couple with at least a portion of the drug delivery device.

26. The accessory as in claim 19, wherein the sensor is configured to detect a movement or a position of the plunger stopper.

27. The accessory as in claim 19, further comprising an actuator operably coupled with the drive assembly, and the sensor is configured to detect a movement or a position of the actuator.

28. The accessory as in claim 18, wherein the feedback indicator includes (a) at least one light source that signifies a movement or a position of a plunger stopper of the drug delivery device, or (b) a plurality of light sources that each signify a movement or a position of a plunger stopper of the drug delivery device.

29. (canceled)

30. The accessory as in claim 28, wherein the feedback indicator comprises (b) the plurality of light sources that each signify a movement or a position of a plunger stopper of the drug delivery device and the plurality of light sources are generally aligned with the window.

31. The accessory as in claim 28, wherein the feedback indicator comprises (b) the plurality of light sources that each signify a movement or a position of a plunger stopper of the drug delivery device and the plurality of light sources provide an injection completion countdown.

32. The accessory as in claim 18, wherein the feedback indicator includes a speaker for generating an audible signal, wherein the audible signal includes (a) an injection completion countdown and/or (b) operation instructions.

33. (canceled)

34. (canceled)
